(19) United States
(12) Patent Application Publication
(10) Pub. No.: US 2008/0234543 A1
(43) Pub. Date: Sep. 25, 2008

(54) SURGICAL DEVICES AND METHOD FOR VAGINAL PROLAPSE REPAIR

(75) Inventor: Steven Goldwasser, Jacksonville, FL (US)

Correspondence Address: MARK YOUNG, P.A., 12086 FORT CAROLINE ROAD, UNIT 202, JACKSONVILLE, FL, 32225, US

(73) Assignee: JJAMM, LLC, JACKSONVILLE, FL (US)

(21) Appl. No.: 12/054,356

(22) Filed: Mar. 24, 2008

Related U.S. Application Data
(60) Provisional application No. 60/896,542, filed on Mar. 23, 2007.
(60) Provisional application No. 60/935,961, filed on Sep. 7, 2007.

(51) Int. Cl. A61F 2/02; A61B 17/42

(52) U.S. Cl. 600/37; 606/119; 623/23.72

(57) ABSTRACT

In accordance with at least one exemplary embodiment, a vaginal prolapse repair procedure can use a perirectal pass in placing an anterior graph arm. For example, the superficial straps of an anterior graft can be place via the transobturator path. The deep straps can be placed via perirectal passes. An exemplary anterior graft can include deep straps that extend from the body of the anterior graft at an angle between about 25 and 60 degrees. Also, an exemplary anterior graft can have a biologic or an absorbable, synthetic strip for providing a window in the implanted graft. An exemplary trocar sheath can be extended and can have a side entry port for cooperating with a trocar. Also, exemplary graspers or retrieval portions of exemplary sheaths can have a reversible locking/mating mechanism for coupling graft arms. Moreover, an exemplary trocar can be provided.

### RELATED APPLICATION

This application claims the benefit of priority of U.S. Provisional Application 60/896,542, filed Mar. 23, 2007, and U.S. Provisional Application 60/935,961 filed Sep. 7, 2007, the entire contents of which are incorporated herein by this reference.

### FIELD OF THE INVENTION

This invention generally relates to vaginal prolapse, and, more particularly, to a minimally invasive surgical procedure, surgical devices and a mesh graft effective in repairing vaginal prolapse.

### BACKGROUND

A common condition suffered by women is prolapse of organs within the pelvic cavity. The organs in a female pelvic cavity—uterus, vagina, bladder and rectum—are held in place by a web of muscles and connective tissues that act like a hammock, collectively referred to as the pelvic floor. When these muscles and tissues that make up the pelvic floor become weakened or damaged, one or more of the pelvic organs may shift out of normal position and fall against the vagina. As a result, the organs press against the vaginal walls to create a hernia-like bulge causing discomfort, affecting bowel and/or bladder function, sexual activity and limiting physical activity. Childbirth is the most common cause of damage to the pelvic floor, particularly where prolonged labor, large babies and instrumental deliveries were involved. Other factors can include past surgery such as hysterectomy, lack of estrogen due to menopause, and conditions causing chronically raised intra abdominal pressure such as chronic constipation, coughing, heavy lifting and other physical activity involving impact with the body. Specific prolapse conditions include cystocele, which is a prolapse of the bladder, rectocele or rectal prolapse, uterine prolapse, and enterocele-post hysterectomy protrusion of the intestines into the vaginal vault (apex).

Vaginal surgery is the usual method of repair, but abdominal surgery (typically open or laparoscopic paravaginal repair, sacralcolpopexy, uterosacral vaginal vault suspension) may also be performed. Traditional pelvic floor repair surgeries, whether abdominal or vaginal, involve lifting the prolapsed organ to restore it back to its correct anatomical position, and subsequently using sutures attached to ligaments and/or muscles to retain the organ in the correct position. Surgeons have also been known to place a layer of mesh below the prolapsed organ, and to subsequently suture corners or sides of the mesh to ligaments or muscles on the sidewalls of the pelvis. The suturing can be done via access through the abdomen or by access through a vaginal incision.

The Gynecare Prolift™ Pelvic Floor Repair System from Gynecare Worldwide, a division of Ethicon, Inc., which is a Johnson & Johnson company, is an example of one present repair system for total pelvic floor repair, anterior pelvic floor repair and posterior pelvic floor repair. Such pelvic repair surgery is performed through the vagina with minimally invasive techniques. During the procedure, a surgeon repositions the prolapsed organs by supporting them with an underlying synthetic mesh which is held in place by its attachment to the surrounding tissues and ligaments in the pelvis. The mesh has a supportive base and a plurality of support arms, which is all held in place by friction between the tissue and support arms. Similarly, U.S. Pat. No. 7,131,943 (Kammerer), U.S. Patent Publication No. 2004/0267088 (Kammerer), and U.S. Patent Publication No. 2004/0039453 (Anderson et al.) each teach a mesh, a surgical kit including a mesh, and a method for using the same to restore a prolapsed organ within a patient's pelvic cavity.

While known prior art procedures, support structures and surgical tools for pelvic floor repair have proven effective for their intended purposes, there is room for improvement. One aspect in need of improvement is placement of the support arms, such that the base is properly configured and positioned to better support the prolapsed organs. In many cases, the apical (deep) support arms of the mesh are not positioned high enough in the pelvis to obtain the optimal support of the anterior apical vaginal wall. When the graft arms are placed in this more optimal location, the existing graft does not adequately conform to the patients anatomy, thus resulting in inadequate support of the apical defect. What is needed is a surgical method, support mesh and surgical tools that facilitate proper positioning of the apical support arms of a pelvic floor repair mesh This will achieve a better anatomic and functional repair of the anterior apical vaginal wall.

The invention is directed to overcoming one or more of the problems and solving one or more of the needs as set forth above.

### SUMMARY OF THE INVENTION

To solve one or more of the problems set forth above, in an exemplary implementation of the invention, surgical devices, a mesh graft and a method for vaginal prolapse repair are provided. According to at least one exemplary embodiment, a vaginal prolapse repair procedure uses a perirectal pass to place an anterior apical graph arm. For example, the superficial straps of an anterior graft can be placed via a transobturator path. The deep straps 710, 715 may be placed via perirectal passes.

An exemplary anterior graft can include deep straps 710, 715 that extend from the body of the anterior graft at an angle between about 25 and 60 degrees. Also, an exemplary anterior graft can have a biologic or an absorbable, synthetic, strip for providing a window in the implanted graft.

An exemplary trocar sheath can be extended and can have a side entry port for cooperating with a trocar. Also, exemplary graspers or retrieval portions of exemplary sheaths and or guides may have a reversible locking/mating mechanism for coupling graft arms. Moreover, an exemplary trocar can be provided in accordance with at least one exemplary embodiment.

An exemplary method for placement of a synthetic graft, having a body and a pair of superficial straps each attached at one end to the body and a pair of deep straps, for correction of vaginal prolapse in a female patient is provided. The method includes steps of bilaterally placing the superficial straps via a transobturator path, proximal to the female patient's bladder neck, and bilaterally placing the deep straps via bilateral perirectal passes to the female patient's ischial spines The step of bilaterally placing the deep straps via bilateral perirectal passes to the female patient's ischial spines includes making two perirectal incisions about two fingers lateral and about two fingers inferior to the female patient's external anal sphincter, bilaterally, and palpably detecting the female patient's ischial spines from within the vagina. A curved piercing tool (e.g., cannula) is guided through each perirectal incision, perirectal fat and through the female patient's levator muscle complex, exiting out of the levator muscle at the base of the ipsilateral ischial spine. Alternatively, the deep straps may be placed through the patient's sacrospinous ligaments through the same method as described above.

An exemplary vaginal prolapse repair sheath for use with a trocar having a curved shaft and pointed tip with a first length is also provided. The sheath includes an elongated, flexible, hollow tube, with an interior diameter greater than an exterior diameter of the curved shaft of the trocar; a second length greater than the first length; an open distal end; an open proximal end with a collar; and an intermediate side entry port configured to receive the pointed tip and curved shaft of the trocar. The intermediate side entry port configured to receive the pointed tip and curved shaft of the trocar being a distance from the open distal end that is less than the first length. Optionally, a resilient valve covering the side entry port and configured to be closed when undisturbed and urged open when the pointed tip and curved shaft of the trocar is inserted may be included. By way of example, the intermediate side entry port configured to receive the pointed tip and curved shaft of the trocar may be a distance from the open distal end that is less than the first length, the distance being about 13 to 20 cm from the open distal end. Also as an example, the second length may be about 2 to 15 cm longer than that distance. The sheath may be marked by coloring or with indicia for association with a strap of a mesh graft. Additionally, the sheath may be configured to releasably engage a corresponding coupling on a strap of a mesh graft. By way of example and not limitation, such couplings may include a female ribbed channel configured to receive and frictionally engage a ribbed corresponding protrusion, a female threaded channel configured to threadedly receive and engage a threaded corresponding protrusion, a female socket configured to receive and engage a corresponding ball joint protrusion, a female channel and side slots configured to receive and engage a corresponding tabbed protrusion, a ribbed protrusion configured to frictionally mate with a corresponding female ribbed channel, a threaded protrusion configured to threadedly mate with a corresponding female threaded channel, a ball joint protrusion configured to interlockingly mate with a corresponding female socket, and a tabbed protrusion configured to interlockingly mate with a corresponding female channel and corresponding side slots. The sheath may also include a flexible loop operably coupled to the open distal end.

An exemplary anterior synthetic graft for surgical repair of pelvic prolapse is also provided. The graft includes a biocompatible synthetic material forming a base, a tail extending posteriorly from the base, and a plurality of superficial and deep straps extending from the base. Each of the plurality of superficial and deep straps comprise an elongated flexible extension. The deep straps extend from the body in divergent relation to the superficial straps. The superficial straps are configured for bilateral placement via bilateral transobturator paths, proximal to a female patient's bladder neck. The deep straps are configured for bilateral placement via bilateral perirectal paths, to the level of the female patient's ischial spines (or alternatively the sacrospinous ligaments). The plurality of superficial and deep straps extending from the base forming an X-like pattern. Optionally, a biologic or an absorbable, synthetic, strip or a window may be disposed on the base. The straps may be marked by coloring or displaying indicia for association with a correspondingly marked engagement device.

An exemplary vaginal prolapse repair grabbing apparatus is also provided. The apparatus includes an elongated flexible body with a distal end including a coupling configured to releasably engage a corresponding coupling on a strap of a mesh graft, the coupling of the distal end including a coupling such as of a female ribbed socket configured to receive and frictionally engage a ribbed corresponding protrusion, a female threaded socket configured to threadedly receive and engage a threaded corresponding protrusion, a female socket configured to receive and engage a corresponding ball joint protrusion, a female socket with side slots configured to receive and engage a corresponding tabbed protrusion, a ribbed protrusion configured to frictionally mate with a corresponding female ribbed channel, a threaded protrusion configured to threadedly mate with a corresponding female threaded channel, a ball joint protrusion configured to interlockingly mate with a corresponding female socket, or a tabbed protrusion configured to interlockingly mate with a corresponding female channel and corresponding side slots.

### BRIEF DESCRIPTION OF THE DRAWINGS

The foregoing and other aspects, objects, features and advantages of the invention will become better understood with reference to the following description, appended claims, and accompanying drawings, where:

FIG. 1 provides a transverse view of female pelvic organs for illustrative purposes; and

FIG. 2 provides a view of surface anatomy of the vulva for illustrative purposes; and

FIG. 3 provides a view of a female bony pelvis for illustrative purposes; and

FIG. 4 provides another view of a female bony pelvis for illustrative purposes; and

FIG. 5 provides another view of female pelvic floor muscles for illustrative purposes; and

FIG. 6 provides a transverse view of female pelvic floor muscles for illustrative purposes; and

FIG. 7 is a plan view of an exemplary graft (i.e., mesh implant) in accordance with principles of the invention; and

FIG. 7A is a plan view of another exemplary graft (i.e., mesh implant) in accordance with principles of the invention; and

FIG. 8 is a perspective view of an exemplary curved trocar, exemplary cannula (i.e., sheath), and exemplary retrieval device in accordance with principles of the invention; and

FIG. 9 is a perspective view of an exemplary trocar inserted into a slot of an exemplary cannula in accordance with principles of the invention; and

FIG. 10 is a perspective view of an exemplary retrieval device inserted through the exemplary cannula in accordance with principles of the invention; and

FIG. 11 is a perspective view of exemplary cannula tips configured to releasably engage mating elements on the free ends of exemplary mesh implant arms in accordance with principles of the invention; and

FIG. 11A is a perspective view of grabbers configured to releasably engage mating elements on the free ends of exemplary mesh implant arms in accordance with principles of the invention; and

FIG. 12 is a perspective view of distal and proximal portions of an exemplary cannula with a removable pointed tip and a looped opposite end in accordance with principles of the invention; and

FIG. 13 provides a view of surface anatomy of the vulva with markings to show incisions made for a pelvic floor repair method in accordance with principles of the invention; and

FIG. 14 provides a view of surface anatomy of the vulva with a trocar inserted for a pelvic floor repair method in accordance with principles of the invention; and

FIG. 15 provides a view of surface anatomy of the vulva with a cannula and retrieving device inserted for a pelvic floor repair method in accordance with principles of the invention.

Those skilled in the art will appreciate that the figures are not intended to be drawn to any particular scale; nor are the figures intended to illustrate every embodiment of the invention. The invention is not limited to the exemplary embodiments depicted in the figures or the types of mesh, devices, relative sizes, ornamental aspects, proportions or steps shown in the figures.

### DETAILED DESCRIPTION

Referring to the Figures, in which like parts are indicated with the same reference numerals, various anatomical views and views of exemplary surgical devices and a method for vaginal prolapse repair according to principles of the invention are shown. Aspects of the invention are disclosed in the following description and related drawings directed to specific embodiments of the invention. Alternate embodiments may be devised without departing from the spirit or the scope of the invention. Additionally, well-known elements of exemplary embodiments of the invention will not be described in detail or will be omitted so as not to obscure the relevant details of the invention.

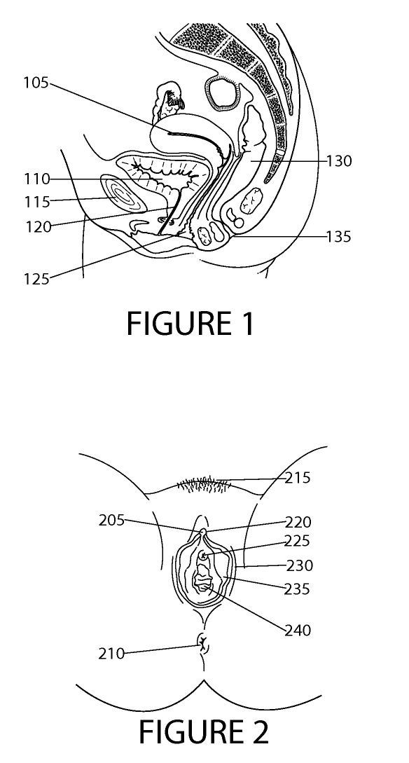

With reference to FIG. 1, a transverse view of female pelvic organs is provided for illustrative purposes. The pelvic organs include the bladder 110, uterus 105 and rectum 130. The vagina 125 is the reproductive tract, i.e., a canal extending from the labia minora to the uterus 105. The pubic symphysis 115 is the midline cartilaginous joint uniting the left and right pubic bones. It is located anterior to the bladder 110 and superior to the external genitalia. The urethra 120 is a tube which connects the bladder 110 to an opening in the vulva between the clitoris and the vaginal opening. The anus 135 is the external opening of the rectum 130.

Now referring to FIG. 2, a view of surface anatomy of the vulva is provided for illustrative purposes. The vulva is the area of the perineum including the mons pubis 215, labia majora 230, labia minora 235 and the openings into both the vagina 240 and urethra 225. The labia minora 235 are two longitudinal cutaneous folds that converge anteriorly to form the prepuce 205 (i.e., hood) of the clitoris 220 and posteriorly to form the fourchette. The anus 210 is the external opening of the rectum 130.

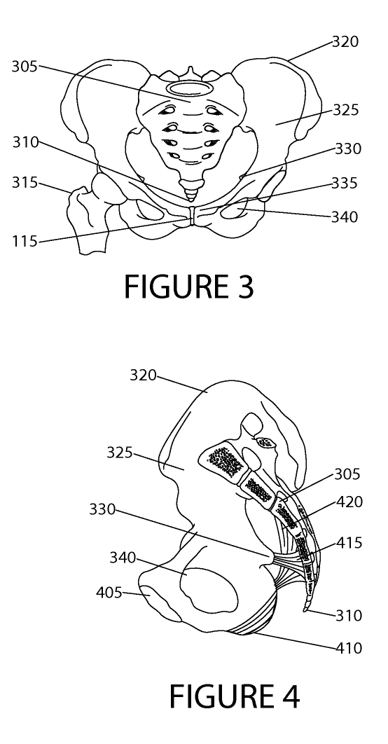

FIGS. 3 and 4 provide views of a female bony pelvis for illustrative purposes. The bony pelvis includes two hip bones (comprised of the ilium 325 and ischium 335) which are joined together by the sacrum 305 posteriorly and by the pubic symphysis 115 anteriorly. The coccyx 310 lies on the inferior aspect of the sacrum 305. The sacrum 305 is a bone at the base of the vertebral column. It attaches to the ilium 325 on the sides. The coccyx 310 is a small vestigial bone that attaches to the base of the sacrum 305. The ilium 325, comprises two large broad plates 325, one on each side of the sacrum 305, each having a curved lateral extremity known as the iliac crest 325. The hip joint of the femur 315 is part of the ilium 325. The ischium 335 are two broad curves of bone, one on each side of the pubic symphysis 115. The body of pubis 405 is the flattened medial portion of the pubic bone entering into the pubic symphysis 115. The rough bony projection at the junction of the lower end of the body of the ischium and its ramus is the ischial tuberosity 410, an attachment for the sacrotuberous ligament 420 and point of origin of the hamstring muscles. The obturator foramen 340 is the hole created by the ischium 335 and pubis body 405 of the pelvis through which nerves and muscles pass.

From the posterior border of the body of the ischium, a thin and pointed triangular eminence known as the ischial spine 330 extends posteriorly. The ischial spine 330 is significant, as it is palpable through the vagina and serves as a target for the locating the deep arms of a mesh graft in an exemplary implementation of a method according to principles of the invention. The sacrospinous ligament 415 is attached by its apex to the ischial spine 330, and medially, to the lateral margins of the sacrum 305 and coccyx 310. The sacrospinous ligament 415 is significant as it is palpable through the vagina and may serve as an alternative (alternative to the base of the ischial spines) fixation point for the deep arms of the mesh graft in an exemplary implementation of a method according to principles of the invention.

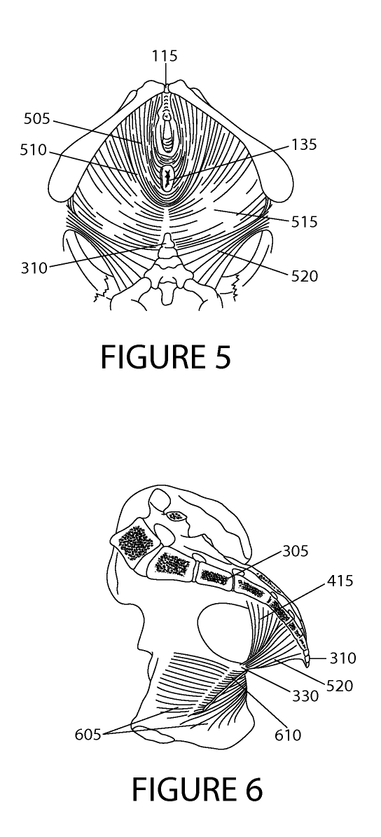

Referring now to FIGS. 5 and 6, views of female pelvic floor muscles are provided. The pelvic floor is a sling of various muscles which are pierced by the urethra, the vagina and the anal canal. The obturator internus muscle 615 sits on the medial side of the ischium 335. The puborectalis muscle 505 forms a sling around the junction of the anus and rectum. The levator ani 610 is a broad thin muscle divided into two parts, the iliococcygeus muscle 515 and the pubococcygeus muscle 510. Together they form the greater part of the floor of the pelvic cavity. The coccygeus 520, a triangular plane of muscular and tendinous fibers, is situated behind the levator ani 610. These muscles support the pelvic organs, holding them in position and resisting exerted forces.

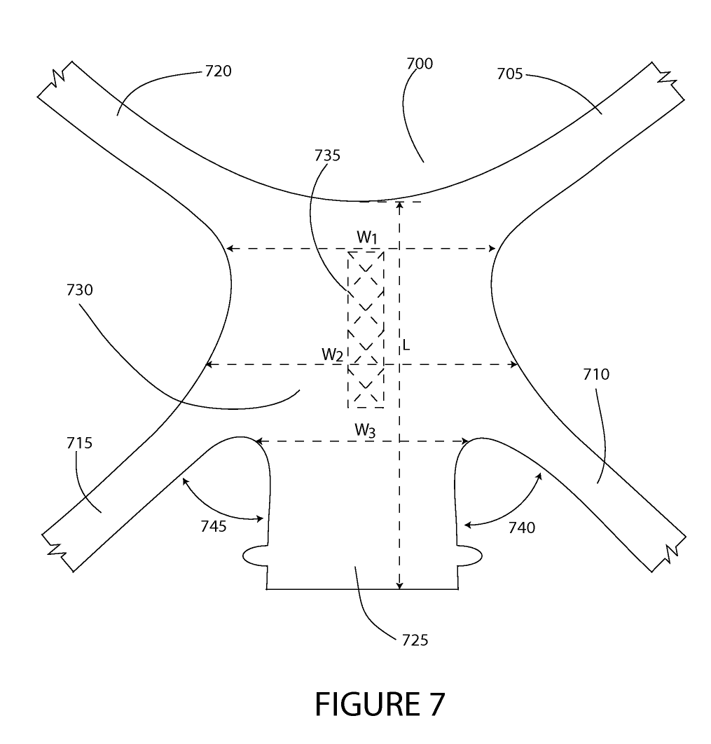

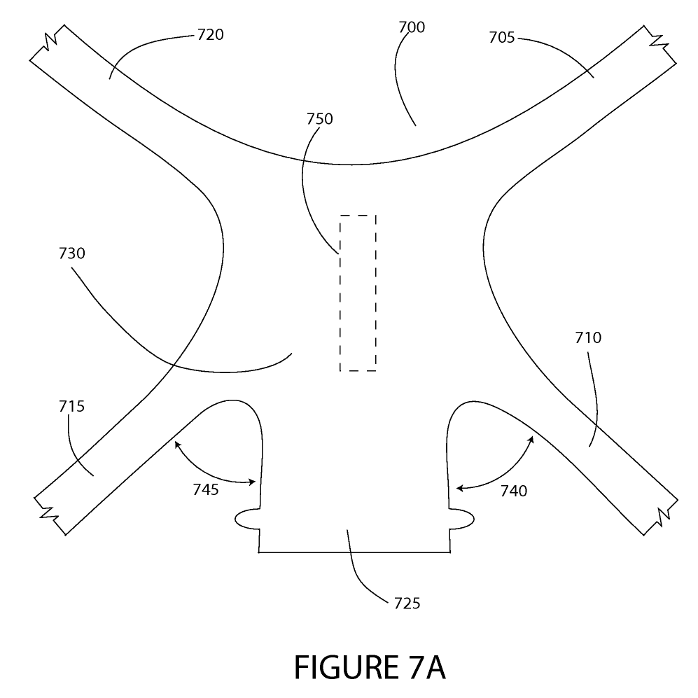

Referring now to FIG. 7, a plan view of an exemplary anterior graft 700 (i.e., mesh implant), which serves as a hammock-like reinforcement for surgical repair of pelvic prolapse by a vaginal approach in accordance with principles of the invention is shown. The anterior graft 700 can be a single structure as shown, or a separate graft attached to a posterior graft as is known to one having ordinary skill in the art. An exemplary graft 700 can be comprised of any tissue-compatible synthetic material, or any natural material, including, but not limited to, autologous, allograft, xenograft, a tissue engineered matrix, or a combination thereof. An exemplary synthetic material is Prolene® polypropylene mesh manufactured by Ethicon. Such an exemplary polypropylene mesh is constructed of knitted filaments of extruded polypropylene.

As shown in FIG. 7, an absorbable biologic or absorbable synthetic, strip 735 can be disposed towards the center of the anterior graft. Other locations for disposing a graft strip 735 will also be appreciated by one having ordinary skill in the art. The graft strip 735 can be made of Vicryl™ manufactured by Ethicon. The graft strip 735 is absorbable by the body and can be absorbed in weeks, unlike the remainder of the graft 700 that can be designed to remain in the body indefinitely. Absorption of the graft strip 735 in the central area leaving a central window in the graft 700 may prevent erosion of the graft 700, as this area along the suture line is more predisposed to exposure of the graft material into the vagina.

Alternatively, as illustrated in FIG. 7A, a window 760 can be disposed in a central area of the graft, which may also prevent erosion of the graft 700, as this area along the suture line is more predisposed to exposure of the graft material into the vagina.

The graft strip 735 or window 760 can be about 1 to 3 cm wide and 4 to 10 cm in length. In at least one exemplary embodiment, the graft strip 735 can be about 2 cm wide and about 6 cm in length. Moreover, the graft strip 735 or window 760 is optional. Similar to background grafts, in certain embodiments, no graft strip 735 or window 760 can be provided.

Prior art anterior grafts are configured for a cutaneous incision for passage of the superficial straps at the anteromedial edge of the obturator foramen, and the cutaneous incision for the deep straps is made adjacent to the superficial incision (e.g., 1 cm lateral and 2 cm below) at the posterolateral edge of the obturator foramen. The result is a passage for the superficial straps adjacent and substantially parallel to or converging upon the passage for the deep straps. Consequently, the straps are spaced close to each other and tend to rumple the base of the graft. Such straps do not provide a broad support framework for the base of the graft because the deep arms of the graft are not placed high enough pelvis to optimally support the anterior apical segment of the vagina.

In a mesh graft 700 according to principles of the invention, two superficial straps (arms) 705, 720 and two deep straps 710, 715 (arms) are provided on the exemplary anterior graft 700. Unlike prior art grafts wherein the superficial and deep straps are spaced apart and substantially parallel or converging, here the deep straps 710, 715 diverge from the superficial straps 705, 720 and extend from the body 730 of the anterior graft 700 at an angle 745, 750 between about 25 and 60 degrees. In at least one exemplary embodiment, an exemplary deep strap 710, 715 can extend from the body 730 of the anterior graft 700 at about a 45 degree angle 745, 750. This unique arrangement of the deep straps 710, 715 relative to the superficial straps 705, 720 provides straps in an anatomical configuration to successfully perform a pass at the anteromedial edge of the obturator foramen for the superficial straps 705, 720 and a perirectal passes for the deep straps 710, 715 in accordance with principles of the invention. The result is a passage for the superficial straps apart and diverging from the passage for the deep straps. Consequently, the straps are spaced wide apart from each other and tend to hold the base of the graft taut. Such an arrangement of straps provides a broad support framework for the base of the graft.

Optionally, one or more of the graft arms 705-720 can be colored or have colored markers, as well as any other type of markers or identification means to distinguish one strap from another. For example, one or more of the straps may be covered by a colored Silastic® sheath. Distinguishing the superficial straps and the deep straps 710, 715 by color (or other added features) can aid in proper identification of proper anatomic placement.

A central segment of the exemplary anterior graft is provided between the two superficial straps 705, 720 and the two deep straps 710, 715. Proximate the superficial straps 705, 720, the central segment may have a width, W₁, of about 7 to 10 cm. In at least one exemplary embodiment, the width can be about 8.5 cm. Proximate the deep straps 710, 715, the central segment can have a width, W₂, of about 9 to 15 cm. In at least one exemplary embodiment, this width, W₂, is about 10.5 cm. These dimensions provide a wider base than that of the conventional prior art mesh graft. The wider base provides superior support.

The anterior graft may also have a posterior tail 725 proximate the deep straps 710, 715. In at least one exemplary embodiment, the posterior tail 725 of the anterior graft 700 extends between the diverging deep straps 710, 715. The posterior tail 725 may have a width, W₃, between about 3 and 5 cm. In at least one exemplary embodiment, the width, W₃, can be about 4 cm. As shown, the length, L, about the central longitudinal axis of the anterior graft can be between 10 and 20 cm. In at least one exemplary embodiment, the length, L, is about 11.5 cm.

Exemplary embodiments of the present invention facilitate increased and more consistent anterior apical support and allow for easier placement of the anterior graft in the correction of anterior apical vaginal prolapse. Exemplary embodiments allow an improved anatomical approach, namely, a perirectal pass-in placing the deep straps 710, 715 of the anterior graph 700. This exemplary approach provides improved anterior apical graft 700 fixation. Additionally, this approach is a significantly easier technique to teach and highly reproducible.

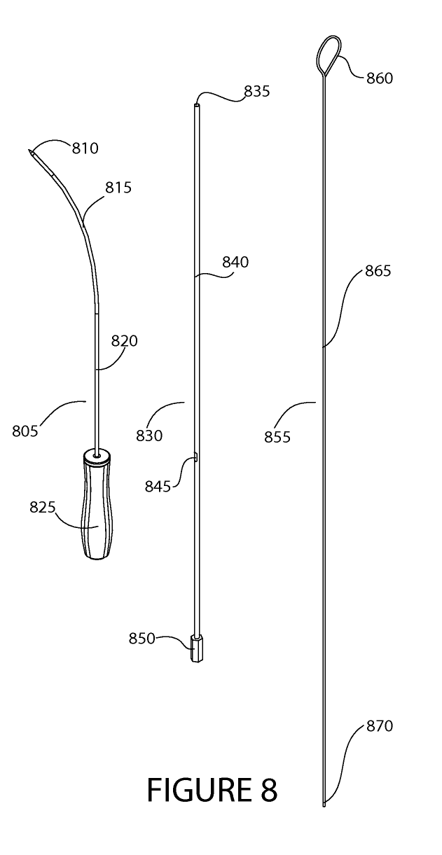

Now referring to FIG. 8, an exemplary trocar 805, sheath (cannula) 830, and looped grasper 855 in accordance with at least one exemplary embodiment of the present invention are shown. A plurality of sheaths 830 may be color coded or otherwise marked for a procedure to correspond to the graft arms of the graft. For example, at least two different colored sheaths 830 can be provided in a kit according to at least one exemplary embodiment of the present invention.

The exemplary sheath 830 of FIG. 8 is an elongated, flexible, hollow tube 840, with an interior diameter greater than the exterior diameter of the shaft 820 of the corresponding trocar 805. The sheath 830 includes an open distal end 835, an open proximal end with a collar 850 to impede inadvertently drawing the end into an incision. The exemplary sheath 830 also includes an intermediate side entry port 845 for receiving the pointed tip and shaft of a trocar 805 that is shorter than the sheath 830. The exemplary sheath 830 may be made of Silastic. The hollow sheath 830 allows the graft arms 705-720 to be pulled through the sheath for positioning. A flexible sheath can allow for the proper placement of the sheath as it is typically manipulated into a nonlinear form. The total sheath length is between about 15 and 35 cm. In at least one exemplary embodiment, the total length is about 30 cm.

The side entry port 845 is a unique aperture defined intermediate to the ends 835, 850 for placing a curved, rigid trocar (guide). Thus, the sheath 830 may be substantially longer than the trocar 805. The additional length facilitates manipulation of the sheath 830 during surgery because it allows for exteriorizing the end(s) 835, 850 of the sheath 830 outside of the patient/operative field, while accommodating a conventional trocar 805.

The side entry port 845 is configured to receive the tip 810, and shaft 820 of the trocar 805. The side entry port can be about 3 to 6 mm long. In at least one exemplary embodiment, the side entry port can be about 4 mm long.

Optionally, a flapper valve may be placed on the port 845. Thus, the side entry port 845 can be a valve-covered hole for placement of the trocar 805. The valve, which can be a resilient flap, allows passage of the trocar through the port 845 but prevents a grasper 855 from passing through the side entry port 845 when being passed through the sheath 830 from the proximal end 850.

The side entry port 845 can begin about 13 to 20 cm from the distal end 835 of the exemplary sheath 830. In at least one exemplary embodiment, the side entry port 845 can begin about 19.5 cm from the distal end 835. In at least one exemplary embodiment, the remaining proximal end 850 can be about 10.5 cm from the port 845.

An exemplary trocar 805 includes a pointed tip 810 defining the distal end, an intermediate curved section 815, and a proximal shaft 820. A handle 825 is attached to the proximal end of the shaft 820. The length of the body 810, 815, 820 of the trocar 805 can be between about 18 to 21 cm. In at least one exemplary embodiment, the length is about 19.5 cm. The diameter of the body 820 can be about 0.1 to 1 mm less than the inner diameter of the tubular body 840 of the sheath 830 for use with the exemplary trocar 805.

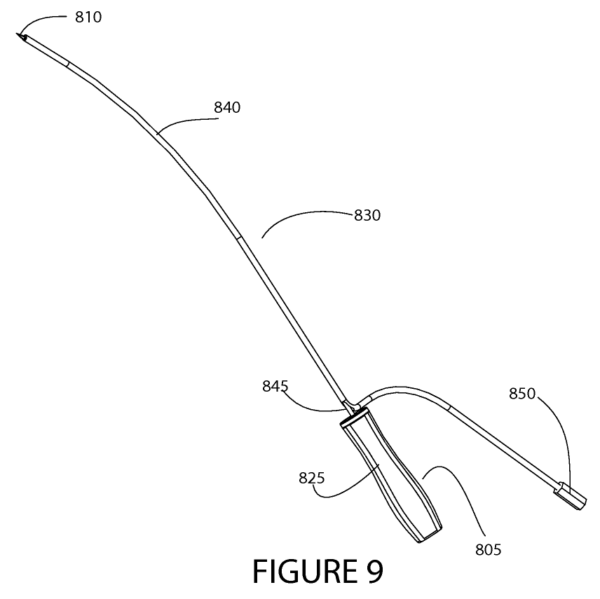

In use with the sheath 830, after an incision has been made, the tip 810 of the trocar 805 is passed through the port 845 and advanced through the tubular body 840 of the sheath 830 until it emerges from the distal end 835 of the sheath 830, as shown in FIG. 9. Thus, the portion of the tubular body 840 of the sheath 830 between the port 845 and distal end 835 receives the curved section 815 and shaft 820 of the trocar 805. While the trocar 805 is installed in the sheath, the tip 810 is designed to enter an incision and pierce tissue as it is advanced into a patient. The path of pierced tissue defines a passage or pass through which strap 705-720 will be drawn. After the pass is completed, the trocar 805 may be withdrawn and removed, leaving the sheath 830 in place in the passage.

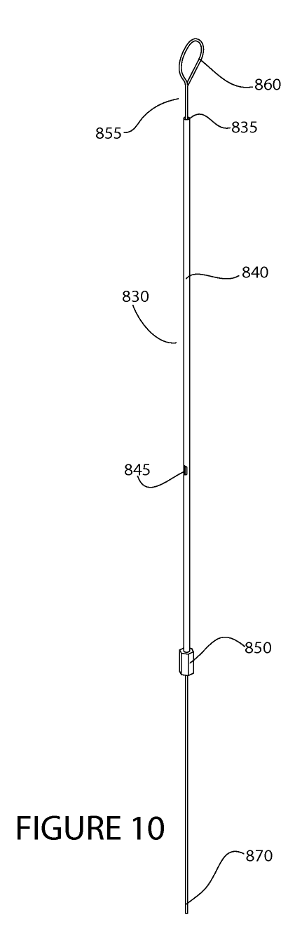

An exemplary grasper 855 is a flexible elongated strand, that is longer than the sheath 830. A loop 860 defines the distal end. An elongated flexible strand 865 defines the body. The loop 860 is configured for receiving and frictionally engaging the free end of a strap 750-720 so that it may be drawn through the sheath 830. The proximal end 870 is designed to remain outside the proximal end of the sheath 850. When the grasper 855 is fed through the sheath 830, as conceptually illustrated in FIG. 10, the loop 860 emerges from the distal end 835 of the sheath 830 while the proximal end 870 of the grasper 855 remains outside the proximal end 850 of the sheath 830. Preferably, a kit includes a sheath 830 and a grasper 855 for each strap 705-720.

A graft arm 705-720 can be folded around or otherwise coupled to the loop 860 and pulled through the flexible sheath 830 into position. The exemplary grasper 830 can be between about 35 and 60 cm in overall length. In at least one exemplary embodiment, the loop grasper is about 50 cm in length. The length (or diameter if circular) of the loop can be between about 2 and 5 cm. In at least one exemplary embodiment, the loop is about 4 cm in length. The body 865 of the exemplary grasper can have a diameter between about 1 mm and 5 mm. In at least one exemplary embodiment, the diameter can be about 2 mm.

As discussed above, while the trocar 805 is installed in the sheath, the tip 810 is designed to enter an incision and pierce tissue as it is advanced into a patient. The path of pierced tissue defines a passage or pass through which strap 705-720 will be drawn. After the pass is completed, the trocar 805 may be withdrawn and removed, leaving the sheath 830 in place in the passage. Then the elongated grasper 855 may be fed through the sheath 830 until the loop 860 emerges from the distal end 835 of the sheath 830 while the proximal end 870 of the grasper 855 remains outside the proximal end 850 of the sheath 830. Thus, loop 860 can be attached to a graft strap 705-720 and then withdrawn through the sheath 830 by pulling back on the free proximal end 870 of the grasper 855.

Therefore, the straps 705-720 of the mesh graft 700 are preferably longer than the elongated sheath 830, so that they may be easily withdrawn. This length of straps 705-720 to accommodate the elongated sheath 830, is another point of dissimilarity between a mesh graft 700 according to principles of the invention and the prior art.

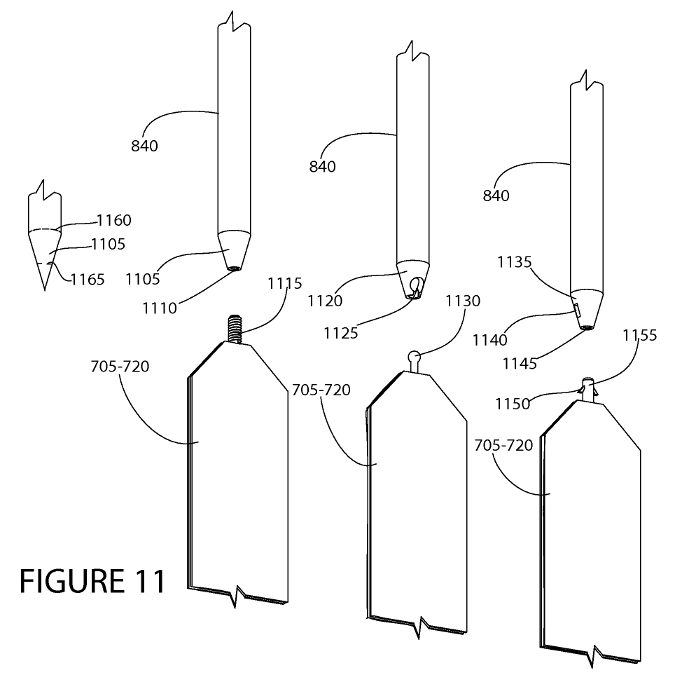

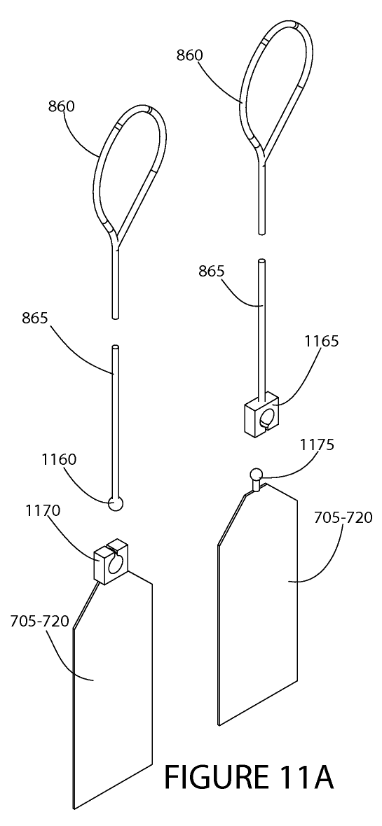

Now referring to FIG. 11, distal portions of additional exemplary sheaths in accordance principles of the present invention are shown. Like the exemplary sheath 830 of FIG. 8, the exemplary alternative sheaths have tubular bodies 840, a side access port 845 and a collar around the proximal end 850. In one embodiment, the remaining distal end 1105 is a hollow or solid pointed tip forming a trocar sheath suitable for piercing penetration of bodily tissue. After a pass is completed with the pointed tip 1105, the pointed tip 1105 may be partially or entirely clipped off, such as at 1160 or 1165, to expose the hollow channel defining the interior of the tube 840 of the sheath 830.

In other exemplary embodiments, the tips 1105, 1120, 1135 have male or female features configured to mate with and releasably engage corresponding female or male mating features attached to the free ends of straps 705-720 of the mesh graft 700. For example, the features may include central apertures 1110, 1125, 1145 and possibly slots 1120, 140, forming female receptacles. Corresponding male mating elements 1115, 1130 or 1150 and 1555 are directly or indirectly attached to free ends of the straps 705-720 of the mesh graft 700. The male or female strap attachment means can be disposed on a plastic (e.g., SILASTIC®) covering for the graft arm. In one embodiment 1105, a ribbed or threaded plug 1115 is configured for frictional engagement in a corresponding central aperture 1110, which may also be ribbed around its interior surface. In another embodiment 1120, a ball socket 1125 is configured for receiving and engaging a ball plug 1130. In yet another embodiment 1135, side slots 1140 are configured for receiving and engaging corresponding resilient tabs 1150 on a stem 1155. In these embodiments, the sheath 830 may act as a grasper (retrieval device), obviating the separate grasper 855. The attachment means may be color coded or otherwise marked to aid in proper anatomic site placement.

Now referring to FIG. 11A, distal and proximal portions of additional exemplary graspers in accordance principles of the present invention are shown. Like the exemplary grasper 855 of FIG. 8, the exemplary alternative graspers have a flexible elongated body 865. The distal ends 1160, 1165 have male or female features configured to mate with and releasably engage corresponding female or male mating features attached to the free ends of straps 705-720 of the mesh graft 700. For example, the features may include a ball 1160 for engaging a socket 1170 on the free end of a strap 705-720. In another embodiment, a ball socket 1165 is configured for receiving and engaging a ball plug 1175 on the free end of a strap 705-720. The ball plugs 1160, 1175 designed to mate with a female socket 1165, 1170 can be between about 1 and 5 mm in length and about 2 mm in diameter. The attachment means may be color coded or otherwise marked to aid in proper anatomic site placement. Additionally, a loop 860 may be provided at the opposite (i.e., proximal end). During surgery, the strap 705-720 may be engaged using either the loop 860 or mating attachment means 1160, 165. Other forms of mating attachments, including, but not limited to, threaded and ribbed corresponding attachment elements may be utilized and are intended to come within the scope of the invention. The mating attachment means 1160, 165 may provide a more reliable fixation and be easier to secure to the straps 705-720 than are the loops 860. If the straps 705-720 are engaged using the mating attachment means 1160, 165, then the loops 1215 may be passed over the mating attachment means 1160, 165 protruding from the vagina, to associate the ends and keep them away from other active surgical sites.

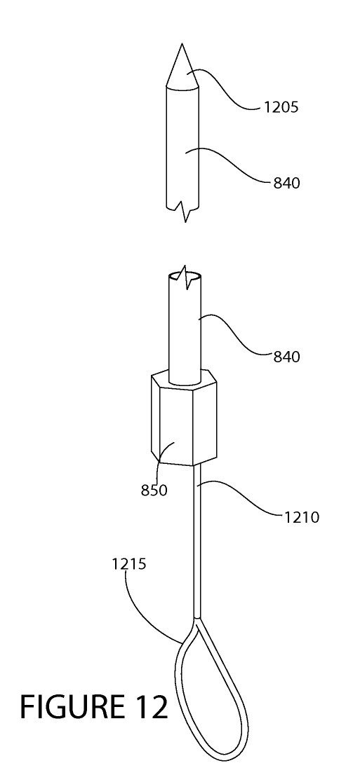

Additionally, with reference to FIG. 12, a loop 1215 may be connected by a resilient lead 1210 to the proximal end 850 of the tubular body 840 of the sheath 830. During surgery, the loop 1215 may be passed over the proximal end of the sheath 830 protruding from the vagina, to associate the ends and keep them away from other active surgical sites.

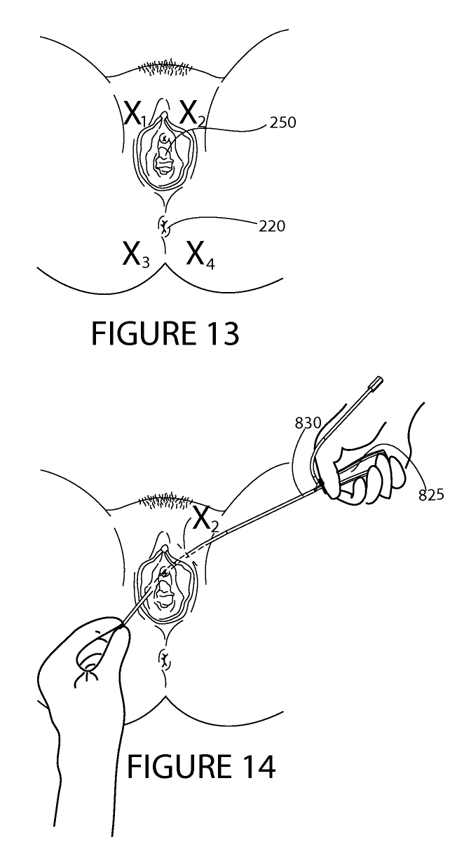

In at least one embodiment, an exemplary procedure involves placement of a graft (synthetic, dermal or a hybrid) for correction of anterior or anterior apical vaginal prolapse. The proximal arms (superficial straps) 705, 720 of the graft 700 can be placed via a transobturator path, proximal to the bladder neck, as is known in the prior art for placement of proximal arms of conventional mesh grafts. The limits of the obturator foramen may be identified by palpation between the thumb and index finger of the obturator membrane where it comes into contact with the bony boundaries. A pair of cutaneous incision for passage of superficial straps 705, 720 of the mesh graft is made in the anteromedial edge of the obturator foramen, at the level of the urethral meatus, denoted by X₁ and X₂ in FIG. 13. The sheath-equipped trocar (as shown in FIG. 9) perforates the obturator extemus muscle and then the obturator membrane. The device should then be advanced medially through the obturator membrane and pass through the obturator internus muscle. A finger positioned inside a vaginal dissection ensures that the device follows a proper path and to protect the bladder, as conceptually illustrated in FIG. 14. Once the distal tip of the trocar and sheath exit the vaginal dissection, the trocar 805 is removed, leaving the sheath 830 in place. The length of the sheath allows the distal end to protrude from the vaginal cavity without interference from the collar 850. This can be performed bilaterally.

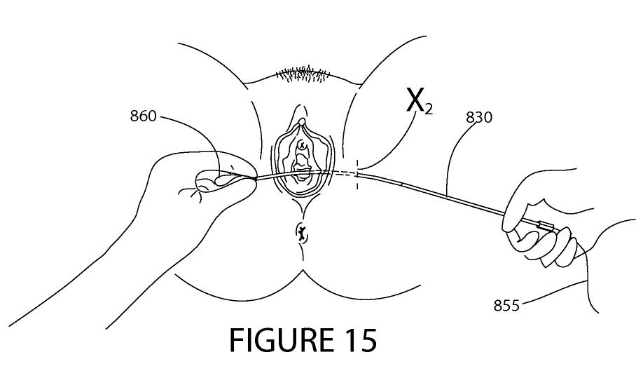

Once the trocar has been removed, the grasper 855 (grasping end first) is advanced through the sheath 830 until the grasping end (e.g., loop 860) emerges out of the distal end of the installed sheath 830. The grasping end is then retrieved through the vaginal dissection and pulled out of the vagina with an instrument or a finger, as conceptually illustrated in FIG. 15.

Rather than use a transobturator path (second transobturator pass) for placing the deep straps 710, 715 like prior art methods, a perirectal pass is followed in an exemplary procedure according to principles of the invention. Two perirectal incisions (1 cm) are made about two fingers lateral and about two fingers inferior to the external anal sphincter 220, bilaterally. The incisions are denoted by X₃ and X₄ in FIG. 13. One of the surgeon's hands can be placed vaginally into the paravaginal space-placing a finger on the patient's ischial spine. With the other hand, the sheath-equipped trocar (as shown in FIG. 9) can be guided through the perirectal incision, perirectal fat and through the levator muscle complex and can exit out of the levator muscle at the base of the ipsilateral ischial spine. Once the distal tip of the trocar and sheath exit the vaginal dissection, the trocar 805 is removed, leaving the sheath 830 in place. The length of the sheath allows the distal end to protrude from the vaginal cavity without interference from the collar 850. This can be performed bilaterally. Alternatively, the sheath-equipped trocar may be guided through the perirectal incision, perirectal fat and through the levator muscle complex and can exit out of the ipsilateral sacrospinous ligament. Once the distal tip of the trocar and sheath exit the vaginal dissection, the trocar 805 is removed, leaving the sheath 830 in place. The length of the sheath allows the distal end to protrude from the vaginal cavity without interference from the collar 850. This can be performed bilaterally.

Once the trocar has been removed, the grasper 855 (grasping end first) is advanced through the sheath 830 until the grasping end (e.g., loop 860) emerges out of the distal end of the installed sheath 830. The grasping end is then retrieved through the vaginal dissection and pulled out of the vagina with an instrument or a finger, as conceptually illustrated in FIG. 15.

After all passes have been made, the distal ends of the straps 705-720 are sequentially coupled to the grasping ends of the graspers (which may be color coded or otherwise correspondingly marked as described above). The graspers are then sequentially pulled through the sheath to the proximal exit. In so doing, the anterior graft is pulled (i.e., drawn) into position over the anterior-apical segment.

The perirectal path followed in at least one exemplary embodiment of the present invention can allows for better (and can be an easier method to perform, teach and reproduce) direct anatomical access to position the graft at the level of the ischial spines or sacrospinous ligaments on all patients. This can result in increased vaginal depth and better graph positioning to address anterior apical (Enterocele or uterine) descent as compared to the prior art systems and methods.

While an exemplary embodiment of the invention has been described, it should be apparent that modifications and variations thereto are possible, all of which fall within the true spirit and scope of the invention. With respect to the above description then, it is to be realized that the optimum relationships for the components and steps of the invention, including variations in order, form, content, function and manner of operation, are deemed readily apparent and obvious to one skilled in the art, and all equivalent relationships to those illustrated in the drawings and described in the specification are intended to be encompassed by the present invention. The above description and drawings are illustrative of modifications that can be made without departing from the present invention, the scope of which is to be limited only by the following claims. Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents are intended to fall within the scope of the invention as claimed.

What is claimed is:

1. A method for placement of a synthetic graft, having a body and a pair of superficial straps each attached at one end to the body and a pair of deep straps, for correction of vaginal prolapse in a female patient, said method comprising steps of bilaterally placing the superficial straps via a transobturator path, proximal to the female patient's bladder neck, and bilaterally placing the deep straps via bilateral perirectal passes proximal to a structure from the group consisting of the female patient's ischial spines and through the female patient's sacrospinous ligaments.

2. A method for placement of a synthetic graft according to claim 1, wherein the vaginal prolapse is a prolapse from the group consisting of anterior prolapsed and anterior apical prolapse.

3. A method for placement of a synthetic graft according to claim 1, wherein the step of bilaterally placing the deep straps via bilateral perirectal passes includes making two perirectal incisions about two fingers lateral and about two fingers inferior to the female patient's external anal sphincter, bilaterally.

4. A method for placement of a synthetic graft according to claim 1, wherein the step of bilaterally placing the deep straps via bilateral perirectal passes includes making two perirectal incisions about two fingers lateral and about two fingers inferior to the female patient's external anal sphincter, bilaterally, and palpably detecting from within the vagina a structure from the group consisting of the female patient's ischial spines and the female patient's sacrospinous ligaments.

5. A method for placement of a synthetic graft according to claim 1, wherein the step of bilaterally placing the deep straps via bilateral perirectal passes at the base of the female patient's ischial spines or sacrospinous ligaments includes making two perirectal incisions about two fingers lateral and about two fingers inferior to the female patient's external anal sphincter, bilaterally, and palpably detecting from within the vagina a structure from the group consisting of the female patient's ischial spines and the female patient's sacrospinous ligaments, and guiding a curved piercing tool through each perirectal incision, perirectal fat and through the female patient's levator muscle complex, exiting out of the levator muscle at the base of a structure from the group consisting of the female patient's ischial spines and the female patient's sacrospinous ligaments.

6. A vaginal prolapse repair sheath for use with a trocar having a curved shaft and pointed tip with a first length, said sheath comprising
an elongated, flexible, hollow tube, with an interior diameter greater than an exterior diameter of the curved shaft of the trocar, and a second length greater than the first length;
an open distal end;
an open proximal end with a collar; and
an intermediate side entry port configured to receive the pointed tip and curved shaft of the trocar.

7. A vaginal prolapse repair sheath according to claim 6, said intermediate side entry port configured to receive the pointed tip and curved shaft of the trocar being a distance from the open distal end that is less than the first length.

8. A vaginal prolapse repair sheath according to claim 6, further comprising a resilient valve covering said side entry port and configured to be closed when undisturbed and urged open when the pointed tip and curved shaft of the trocar is inserted

9. A vaginal prolapse repair sheath according to claim 6, said intermediate side entry port configured to receive the pointed tip and curved shaft of the trocar being a distance from the open distal end that is less than the first length, said distance being about 13 to 20 cm from the open distal end.

10. A vaginal prolapse repair sheath according to claim 6, said intermediate side entry port configured to receive the pointed tip and curved shaft of the trocar being a distance from the open distal end that is less than the first length, said distance being about 13 to 20 cm from the open distal end, and said second length being about 2 to 15 cm longer than the distance.

11. A vaginal prolapse repair sheath according to claim 6, said sheath being marked for association with a correspondingly marked strap of a mesh graft, said sheath being marked using a marking means from the group consisting of a color and an indicia.

12. A vaginal prolapse repair sheath according to claim 6, said open distal end including a coupling configured to releasably engage a corresponding coupling on a strap of a mesh graft.

13. A vaginal prolapse repair sheath according to claim 6, said open distal end including a coupling configured to releasably engage a corresponding coupling on a strap of a mesh graft, said coupling of the open distal end comprising a coupling from the group consisting of
a female ribbed channel configured to receive and frictionally engage a ribbed corresponding protrusion,
a female threaded channel configured to threadedly receive and engage a threaded corresponding protrusion,
a female socket configured to receive and engage a corresponding ball joint protrusion,
a female channel and side slots configured to receive and engage a corresponding tabbed protrusion,
a ribbed protrusion configured to frictionally mate with a corresponding female ribbed channel,
a threaded protrusion configured to threadedly mate with a corresponding female threaded channel,
a ball joint protrusion configured to interlockingly mate with a corresponding female socket, and
a tabbed protrusion configured to interlockingly mate with a corresponding female channel and corresponding side slots.

14. A vaginal prolapse repair sheath according to claim 6, further comprising a flexible loop operably coupled to the open distal end.

15. An anterior synthetic graft for surgical repair of pelvic prolapse comprising a biocompatible synthetic material forming a base, a tail extending posteriorly from the base, and a plurality of superficial and deep straps extending from the base, each of said plurality of superficial and deep straps comprising an elongated flexible extension, said deep straps extending from the body in divergent relation to the superficial straps, said superficial straps being configured for bilateral placement via bilateral transobturator paths, proximal to a female patient's bladder neck, and said deep straps being configured for bilateral placement via bilateral perirectal paths, at the base of a structure from the group consisting of the female patient's ischial spines and the female patient's sacrospinous ligaments.

16. An anterior synthetic graft for surgical repair of pelvic prolapse according to claim 15, wherein the plurality of superficial and deep straps extending from the base forming an X-like pattern.

17. An anterior synthetic graft for surgical repair of pelvic prolapse according to claim 15, further comprising a biologic or an absorbable, synthetic strip disposed on the base.

18. An anterior synthetic graft for surgical repair of pelvic prolapse according to claim 15, further comprising a window disposed on the base.

19. An anterior synthetic graft for surgical repair of pelvic prolapse according to claim 15, wherein each of said plurality of superficial and deep straps is marked for association with a correspondingly marked engagement device, said straps being marked using a marking means from the group consisting of a color and an indicia.

20. A vaginal prolapse repair grabbing apparatus comprising an elongated flexible body with a distal end including a coupling configured to releasably engage a corresponding coupling on a strap of a mesh graft, said coupling of the distal end comprising a coupling from the group consisting of
a female ribbed socket configured to receive and frictionally engage a ribbed corresponding protrusion,
a female threaded socket configured to threadedly receive and engage a threaded corresponding protrusion,
a female socket configured to receive and engage a corresponding ball joint protrusion,
a female socket with side slots configured to receive and engage a corresponding tabbed protrusion,
a ribbed protrusion configured to frictionally mate with a corresponding female ribbed channel,
a threaded protrusion configured to threadedly mate with a corresponding female threaded channel,
a ball joint protrusion configured to interlockingly mate with a corresponding female socket, and
a tabbed protrusion configured to interlockingly mate with a corresponding female channel and corresponding side slots.
